(19) United States
(12) Patent Application Publication
(10) Pub. No.: US 2011/0076191 A1
(43) Pub. Date: Mar. 31, 2011

(54) Ozone Based Method and System for Tool Sterilization

(75) Inventors: Eugene I. Gordon, Mountainside, NJ (US); Sean Michael Reilly, East Stroudsburg, PA (US)

(73) Assignee: GERMGARD LIGHTING, LLC., Dover, NJ (US)

(21) Appl. No.: 12/567,131

(22) Filed: Sep. 25, 2009

(51) Int. Cl. A61L 2/20; A61L 2/16

(52) U.S. Cl. 422/29; 422/292; 422/291; 422/177

(57) ABSTRACT

A method and system for sterilizing an item using ozone and a secondary molecule is provided. The secondary molecule comprises hydrogen for conversion to a hydroxyl radical. The item and secondary molecular are inserted into a container, and a gaseous mixture containing ozone can be inserted into the container by way of a filling station having an ozone source and a vacuum pump to remove air from the container. Sterilization occurs within the container such that the container can be removed from the filling station. A preferred secondary molecule is an alcohol, such as isopropyl alcohol. Prior to insertion of the item into the container, the item can be soaked in a nutrient rich hot water bath at or near boiling and optionally at a pressure above atmospheric to assist in sterilization.

### FIELD OF THE INVENTION

The present invention relates to sterilization of tools, and more particularly to a method and system for sterilizing surgical instruments and tools utilizing ozone and a secondary molecule having available hydrogen for producing oxidizing radicals.

### BACKGROUND

Surgical instrument sterilization is a critical step preceding any surgery, whether in a civilian hospital, small surgical facility, a military field hospital, or under emergency conditions. Sterilization requires a reduction in the initial number of any type of active pathogens in any arbitrary area of the surgical instrument by a factor of 10⁶ (i.e., −6 log₁₀ reduction) or, in other words, inactivation of 99.9999% of the initial number of active pathogens of that type in that area. The current rules and regulations for a newly manufactured instrument, and reuse of a previously used instrument, require sterilization of the instrument. Moreover, since surgical instruments are generally costly, it is becoming increasingly desirable to repair or recondition, clean, sterilize, and repackage previously used surgical instruments for reuse.

Presently, newly manufactured surgical instruments are sealed in a sterility-preserving pouch or package which is then sent for sterilization to a central gamma radiation facility. This procedure incurs considerable expense and lost time. Alternative sterilization systems, which are generally used for re-sterilizing used instruments, include high temperature steam in autoclaves, the most common system, and room temperature systems, ethylene oxide gas, (ETO), vaporized hydrogen peroxide gas, (VH₂O₂), and ozone/water vapor, (O₃/H₂O).

Although autoclaves are the most commonly used surgical instrument sterilization system, they have serious disadvantages. For example, autoclaving causes significant degradation of surgical instruments and, therefore, usually only stainless steel surgical instruments can be sterilized in this manner. However, even stainless steel requires overhaul of the instrument after a number of uses. Furthermore, high throughput autoclaves require steam generators, substantial volumes of water, and high electric power capability. The autoclave process requires at least 15 minutes in a shortcut mode, and more frequently well over an hour to complete.

Room temperature, gas systems require lengthy processing. ETO requires a 15½-hour cycle and is poisonous and explosive. A VH₂O₂ system requires up to 60 minutes of exposure, but has been deemed inadequate by manufacturers of endoscopes and other tools having an internal volume, because the internal volumes are difficult for the sterilizing gases to reach. An O₃/H₂O system has a long 4½-hour cycle and has recently been approved for endoscopes, but the equipment can be prohibitively expensive and few are in use. Small autoclaves, such as those used in dental offices, doctors' offices, veterinarian offices, laboratories, and other small operating facilities are costly and sometimes prohibitively expensive. Large autoclaves are used in military field hospitals and are difficult to transport and use too much water and electric power. Gamma radiation systems for sterilization of new instruments cost millions of dollars.

Improvements relative to current sterilization methods and systems are clearly desirable.

### SUMMARY OF THE INVENTION

In accordance with one aspect of the present invention, a method and system for sterilizing a surgical instrument or other item is provided. One or more containers are used to store the items being sterilized. A secondary molecule comprising hydrogen (e.g., liquid alcohol) is placed or injected into the sterilizing volume creating a vapor within the container and a gaseous mixture including ozone then fills the container and interacts with the secondary molecule vapor creating hydroxyl and related radicals within the container. The container is then closed off, so as to trap the ozone gas and secondary molecule vapor within the container. The ozone quickly interacts with and converts the secondary molecule vapor to the oxidizing radicals and some byproducts of the secondary molecule. The residual ozone concentration becomes negligible and the radicals then sterilize the item inside the sealed container.

Prior to filling the container with the ozone and oxygen mixture, the container can be evacuated of the air within by a vacuum pump. The gaseous mixture subsequently introduced into the container can include ozone and oxygen, and preferably comprises approximately 7% ozone. In a further feature of the present invention, the secondary molecule is an alcohol. One preferred alcohol is isopropyl alcohol. The secondary molecule can be inserted into the container via an absorbent material impregnated with the secondary molecule. Alternatively, the filling station can be used to inject a secondary molecule vapor into the container. A measured amount of alcohol evaporates so there is no liquid alcohol left.

Prior to insertion of the item being sterilized in the container, the item may be soaked in a hot water bath to assist in sterilization. The hot water bath can be boiling (i.e., 100 C) or near boiling (e.g., about 95-100 C). Additionally, the water bath can be under a higher pressure, for example one to ten atmospheres. Various nutrients can also be included in the water bath to encourage activation and germination of any bacterial endospores on the item being sterilized. Vegetated bacteria are more readily inactivated than endospores.

The sterilizing container further includes means to connect to a vacuum pump and evacuate the residual ozone and other gases and vapors, leaving a partial vacuum in the container. A catalytic converter before or after the vacuum pump can be outfitted with a carbon catalyst that converts the ozone to oxygen as the gases inside the container are evacuated.

These and other advantages of the invention will be apparent to those of ordinary skill in the art by reference to the following detailed description and the accompanying drawings.

### BRIEF DESCRIPTION OF THE DRAWINGS

FIG. 1 is a diagram of a system in accordance with an embodiment of the present invention; and

FIG. 2 is a flow diagram of a process in accordance with an embodiment of present invention.

### DETAILED DESCRIPTION

In accordance with the present invention, sterilization is achieved using ozone in combination with a secondary molecule that includes hydrogen (i.e., a hydrogen moiety) for reaction with the ozone to produce hydroxyl and related radicals. Such secondary molecules include alcohol but can include a wide variety of molecules (e.g., ammonia, water, and hydrogen gas). One gaseous secondary molecule that has been found to be particularly effective and practical is isopropyl alcohol (“IPA”), in part because it is easily available in a liquid state at atmospheric pressure and is readily converted to a vapor state in the container.

This combination of ozone and a secondary molecule described above, when used in accordance with the present invention, has been demonstrated to sterilize items in the container in no more than three minutes using the standard test spore, Geobacillus stearothermophilus. Complete inactivation of all pathogens including spores and prions is also achievable. Additionally, because the present invention achieves sterilization within a sealed or closed, lightweight and portable container or kit that is filled with the sterilizing mixture, the throughput of items sterilized can be greatly increased. While the item is being sterilized in the container, another container storing additional items to be sterilized can be prepared and filled through another set of connection means at the filling station. Alternately, a number of pouches containing one or more items can be filled in parallel leaving the sterilized item in a sealed pouch. Sterilization of a set of items does not occur within the filling station or otherwise require sole engagement and use of the filling station. The number of parallel kits that can be processed is determined by the capacity of the filling station. The instruments within the kit can be used immediately upon the second evacuation, providing a sterilization cycle lasting no more than 3 minutes.

Alternatively, the kits do not need to remain connected to the filling station during sterilization. Rather, once a first container is filled with the mixture from the filling station, it can be removed from the filling station. While the item inside the first container is being sterilized by the gases trapped in the container, a second kit can be filled at the filling station, thus allowing for further parallelization of the sterilization and kit preparation process.

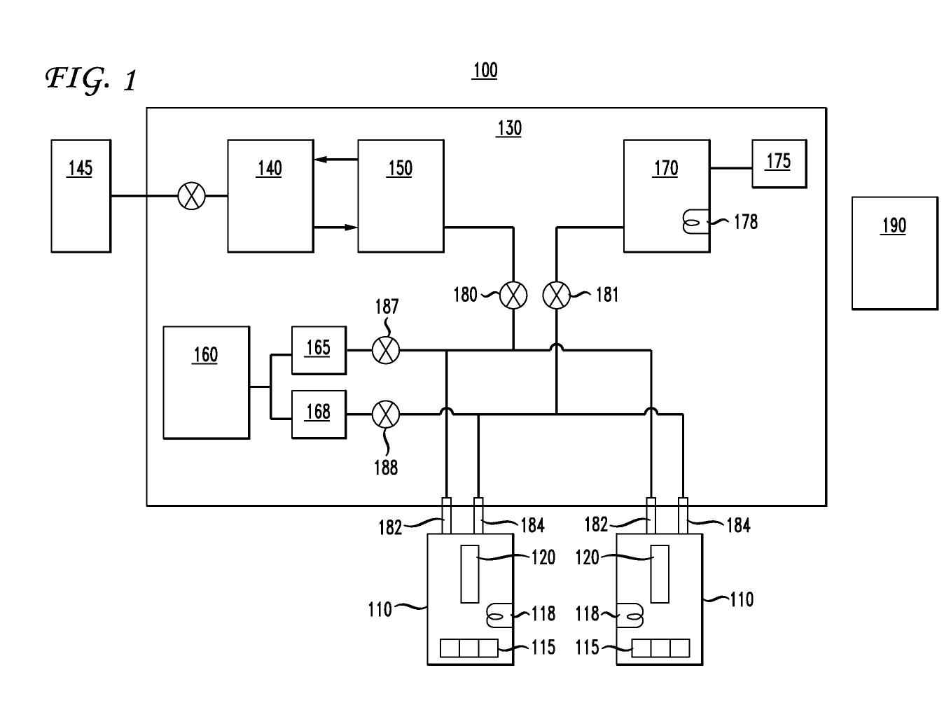

With reference to the Figures, FIG. 1 illustrates a system 100 in accordance with an embodiment of the present invention that provides a safe, inexpensive, and efficient ozone-based system and method for rapidly sterilizing items such as surgical instruments and tools. The system 100 includes a filling station 130 that can be connected to a container 110 storing items such as tools or instruments 120. For example, an entire kit of tools required for an operation or other medical procedure can be sterilized and stored in a container for on demand use during surgery. However, while the following description focuses primarily on the sterilization of medical instruments, one of ordinary skill in the art would recognize that the present invention can be utilized with a variety of items that can fit within an appropriately sized container 110 and are not adversely affected by ozone or other chemicals used in the sterilization process.

The filling station 130 includes an ozone source such as an ozone generator 140 that receives a source of oxygen 145 and converts the oxygen to ozone. The ozone generator 140 outputs ozone and oxygen that has not been converted to ozone by the ozone generator. One inexpensive and lightweight method of producing ozone is a corona discharge in the presence of oxygen. Alternatively, a vacuum ultra-violet (VUV) based system can be used to produce the ozone and oxygen mixture. This resulting gaseous mixture can be stored in a storage tank 150 to provide on-demand service from the filling station. Alternatively, rather than providing an ozone generator 140 within the filling station 130, ozone can be generated external to the filling station 130 and connected to the filling station 130. In a further alternative, storage tank 150 can be filled external to the filling station 130 and connected to a filling station 130 as an interchangeable supply source.

The filling station 130 also includes a vacuum pump 160. The vacuum pump can be used to remove the gas from the container 110 prior to introducing the ozone mixture into the container 110 and later evacuating the container after the sterilization is complete. A number of valves are included to control the flow of gas within, into, and out of the filling station 130. The container 110 can be evacuated and filled via valve connectors 182 and 184 to the filling station 130.

Each container 110 connects to the filling station 130 via valve connectors 182 and 184 to remove and add gas from the filling station. The container 110 can also include a second connection to an ozone concentration meter to assure the proper ozone concentration at the beginning and end of the sterilization cycle. Thus, the container can connect to the filling station and vacuum pump 160 via the connectors 182 and 184, which control the flow of gas into and out of the container. Other connection arrangements are possible and would be known to a person of ordinary skill in the art.

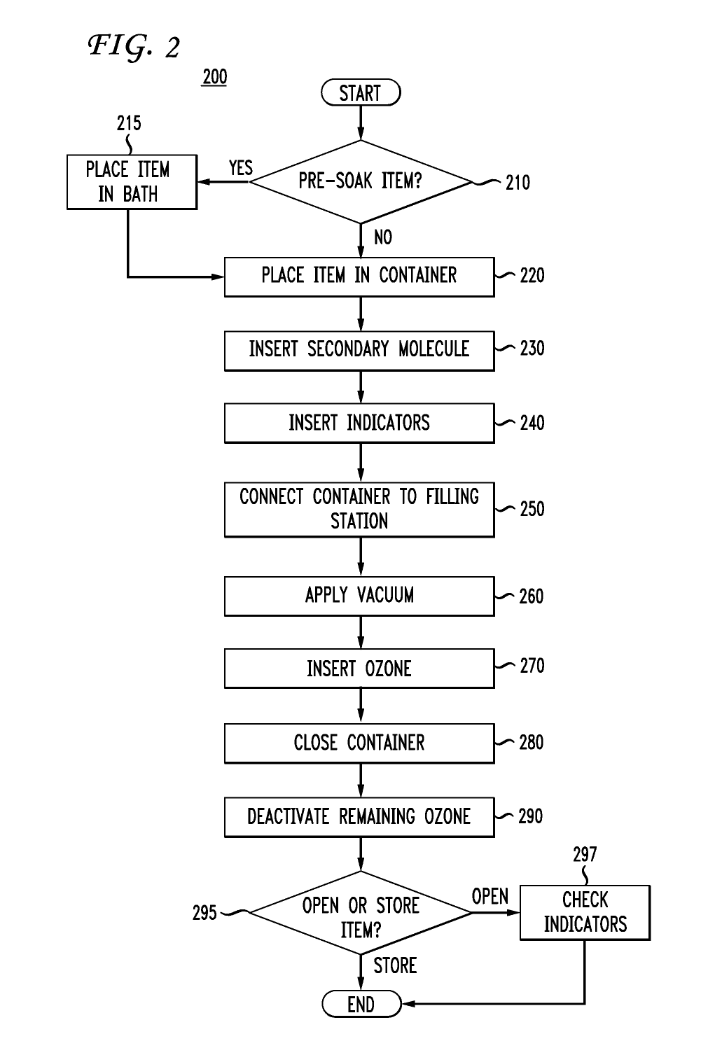

In operation, the system 100 is used to sterilize items as illustrated in FIG. 2 by process 200. In accordance with this process, a determination is made as to whether or not to pre-soak the item at step 210. If a pre-soak is desired, the item is placed in the pre-soak bath 190 at step 215. If pre-soaking is not desired or not necessary, the item is placed in a container 110 at step 220. The pre-soak procedure and the benefits thereof are discussed in further detail below.

The secondary molecule is inserted into the container 110 at step 230. In accordance with an embodiment of the present invention, the filling station 130 can include a supply of the secondary molecule in the storage unit 170 which is used to inject the secondary molecule vapor into the container 110. As illustrated, the secondary molecule is stored in liquid form in storage unit 170. A heating element 178 is provided to heat the liquid secondary molecule and convert a portion of the secondary molecule into a vapor. A pressure gage 175 measures the pressure of the vapor secondary molecule in storage tank 170 and controls the temperature of the heating element 178 to adjust the pressure of the secondary molecule vapor in the storage tank 170. In accordance with one feature of the present invention, the partial pressure of the secondary molecule within the container is between 20 and 100 mm of mercury.

The secondary molecule is preferably kept separately from the ozone and prevented from reacting with the ozone within the filling station by providing a separate ozone valve/connector 182 and secondary molecule valve/connector 184 for each container 110. Additionally, flow of the ozone and secondary molecule vapor can be controlled by valves 180 and 181. Alternatively, the secondary molecules can be inserted into the container 110 using the same connection that inserts ozone into the container. However, valves 180 and 181, or other means, preferably prevent the ozone from mixing (i.e., reacting) with the secondary molecule outside of the container 110.

In accordance with a further embodiment of the present invention, the secondary molecule is added to a gauze pad which is inserted into the container 110. Alternatively, a fibrous cloth that is already impregnated with the secondary molecule is added to the contents of the container 110. For example, a pad pre-moistened with the secondary molecule and included in a sealed package is opened and the pre-moistened pad is inserted into the container 110. In a further embodiment, a strip 115 is integrated into the container 110. The strip 115 can include an absorbent portion onto which the secondary molecule can be added. Alternatively, the strip 115 can be pre-moistened and sealed to the container such that a user can tear open a protective seal to expose the pre-moistened pad.

A chemical indicator, one of many possible types, is added to the container at step 240. For example, a chemical indicator that changes color in the presence of the oxidizing radicals can be included so that a user can visually verify that the sterilization process has occurred. Additionally, an ozone monitor can be attached to the container that indicates the pressure of ozone. Thus, if the ozone-concentration indicator does not show reduced ozone concentration, the user will know that the ozone within the container 110 has not yet converted to oxygen and therefore sterilization may not have been completed. Each chemical indicator can be provided in its own strip that is integrated into the container 110 or added manually. Alternatively, the indicators can be included in strip 115, such that unsealing of strip 115 results in the addition and exposure of the secondary molecule as well as the addition and exposure of the various chemical indicators.

The container 110 is then connected to the filling station 130 at step 250. The vacuum pump 160 then applies a vacuum to the rigid container to remove the air from the container 110. The required strength of the vacuum pump 160 varies depending in part on the desired vacuum to be achieved in the container 110 and the time dedicated to achieving the vacuum.

As the pressure of the container 110 is reduced, the evaporation of the secondary molecule increases. As discussed above, while any simple or complex alcohol capable of being vaporized into the vapor state can be used as a secondary molecule, molecules such as ethylene glycol and propylene glycol that contain more than one hydroxyl group could be used to increase the efficiency of the hydroxyl radical formation. This increased efficiency is due to the presence of multiple hydroxyl groups in the molecule that could be converted to hydroxyl radicals in the presence of ozone.

Additionally, it should be noted that more complex molecules may produce toxic byproducts (such as aldehydes or ketones) that settle on the instruments during the ozone oxidation process, which would need to be removed from the sterilized items prior to use to avoid patient contamination. Alcohols having a relatively small carbon chain (e.g., less than four carbon atoms) limit the likelihood of toxic byproduct formation during sterilization. Thus, advantageous secondary molecules include methanol, ethanol, isopropanol, and butanol.

After the air is removed from the container at step 260, the ozone and oxygen mixture in the storage tank 150 is injected into the container 110 at step 270. Various concentrations of ozone can be used. However, in an advantageous embodiment, the partial pressure of the ozone is about seven and one half percent. The container 110 can then be closed at step 280. The tools and secondary molecule were previously sealed or closed within the container such that the point of influx to the container 110 is through the connector and valves.

The sterilization process occurs through the oxidation of the biological agents on the surface of the items 120. Oxidizing agents (i.e., oxidizers) are atoms, molecules, or ions that are capable of accepting one or more electrons from a differing atom, molecule, or ion. Ozone is an efficient oxidizer and is a particularly effective in inactivating Giardia and Cryptospiridium. However, certain molecules have an even stronger oxidation potential. Two such molecules are the hydroxyl radicals OH and O₂H. Due to the incomplete electron shell of this molecule, hydroxyl radicals are inherently unstable and attract electrons to complete a stable octet electron shell. There are two possible ways (e.g., reactions) that the hydroxyl radical can attain a stabilizing octet electron shell. A first reaction is oxidation, defined as follows:

OH+R [symbol] OH⁻+R⁺ [1]

The second reaction is hydrogen abstraction, which is defined as follows:

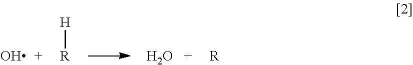

In the above equations, R represents the reductant molecule (i.e., a substance capable of bringing about the reduction of another substance as it itself is oxidized) that is undergoing oxidation by the hydroxyl radical. From these equations, it can be seen that hydrogen abstraction is a type of oxidation reaction, where an electron is transferred from the reductant to the oxidizer. Moreover, in hydrogen abstraction, a hydrogen atom is additionally transferred from the reductant to the oxidizer.

To disinfect surgical instruments (e.g., items 120), ozone is utilized with a secondary molecule (such as hydrogen or alcohol) to generate the highly efficient oxidizing hydroxyl radical. This molecule reacts with the pathogens on the surgical instruments and acts to change their chemical make-up to render these pathogen molecules harmless to humans. The organic pathogen molecules are laden with areas of delocalized electrons. Delocalized electrons are electrons that are not directly associated with a sigma (single) bond. Delocalized electrons can be in the form of pi (double or triple) bonds or unbound electrons. Chemical moieties (i.e., a specific segment of a molecule (e.g., aniline and ethidium bromide each have a phenyl and an amino moiety)), that enable delocalized electron populations are shown below in Table 1.

TABLE 1
Chemical moieties that enable delocalized electrons

| Moiety | Chemical Structure |
|---|---|
| Double Bond | C═C |
| Triple Bond | C≡C |
| Carbonyl | 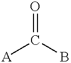 |
| Carbonate | 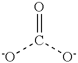 |
| Carbamate | 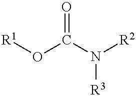 |

All of the above chemical moieties can be found in organic pathogens. When the moieties are linked together (such as a carbonyl and a double bond), the amount of delocalized electrons is increased. Delocalization allows for radical stabilization, as the radical can move throughout the delocalized area. When hydrogen is adjacent to or in the area of delocalized electrons, this hydrogen becomes a key site for hydrogen abstraction. Thus, when the hydroxyl radical reacts with the pathogen, some degree of oxidation and some degree of hydrogen abstraction will occur. In both mechanisms, the pathogen is left with a radical in the molecule. This radical formation leads to other reactions, such as chain scission or radical-radical termination. Both reactions lead to the destruction of the native pathogen.

The foregoing oxidation and sterilization processes occur within the container 110 even after it has been removed from the connectors 182 and 184. Thus, while the items 120 in the container 110 are being sterilized, another container 110 can be processed in the manner described above and illustrated by process 200.

Because of the very short sterilization time required by the present invention, the container 110 can be practically immediately brought to a site for use. Alternatively, because the container 110 is sealed and sterilization occurs within the sealed container 110, items 120 have been never been touched by potential contaminants after sterilization. Thus, the container 110 can be stored indefinitely as a sterile kit.

At step 290, the container is preferably evacuated prior to storage or use (e.g., opening). For example, the container 110 can be connected to vacuum pump 160 of the filling station for evacuation of the gases within the container 110. Ozone rapidly converts to oxygen molecules (i.e., O₂) in the presence of heat or when passed through a catalyst 165 such as a carbon filter. Thus, in accordance with one feature of the present invention, the container 110 can include a heating element 118 that can be activated while the container 110 is still sealed to convert any remaining ozone to oxygen. The heating element 118 can include a simple battery powered light bulb or other heating source. Alternatively, a catalyst can be included in a connector or filter (e.g., the exhaust connect 165), and the remaining gas in the container 110 evacuated from the container 110 through the catalyst to convert any remaining ozone to oxygen. While it is preferable that a predetermined amount of secondary molecule is inserted into the container 110 such that no liquid will remain within the container 110, a cooler 168 can be used to condense any secondary molecule vapor and collect the resulting or remaining liquid.

At step 295, it is determined whether the container is to be opened or stored for a period of time. If the container is to be stored, the process 200 ends. However, if the container 110 is to be opened, several precautionary steps should be taken for safety. For example at step 297, if chemical indicators were included in the container, either as part of strip 115 or as separate, standalone additions to the container, the user should check the indicators to determine whether any ozone remains in the container 110 and/or whether any biological contamination of the items 120 in the container 110 has occurred. If at step 297 it is determined that ozone is present in the container 110, or as a prophylactic measure, the user can take further precautions to deactivate the remaining ozone.

While vegetative bacteria and viruses can be inactivated in less than three minutes with exposure to ozone and a secondary molecule, some contaminants, such as spores, are very challenging to deactivate due to their tough outer shell. The tough outer shell of a spore makes penetration of the sterilizing gas a slow process. Of the previously known methods of sterilization, only autoclaves and gamma radiation systems readily inactivate spores. Thus, returning to steps 210 and 215, the tools can be pre-soaked to increase inactivation of spores and the like.

Soaking the items 120 in a hot water bath, for example at a temperature above 65 C for thermophile spores, effectively cracks or thins the shell of any spores and converts the spore for a given bacteria into the vegetated state thereby enabling rapid sterilization using the process described above. Thus, In accordance with one aspect of the present invention, if at step 210 it is determined that the items should be pre-soaked, at step 215 the items are placed in a hot water bath for a short period of time. The hot water bath can be a simple bath in boiling water (i.e., 100 degrees Centigrade) or even lower temperatures, such as 97 degrees Centigrade.

Generally, a 15-minute bath in water at a temperature of 95°-100° C. results in spore activation and germination and allows for sterilization of the vegetated bacteria by exposure to ozone and the secondary molecule. The germination process can be accelerated by adding nutrients to the water bath to accelerate germination. The germination process can be further accelerated by pressurizing the boiling water (e.g., up to 10 atmospheres).

Returning to the issue of selecting a secondary molecule, it is noted that alcohol efficiently produces oxidizing radicals in the present of ozone. Isopropanol is one such alcohol that is colorless, flammable, chemical compound with a strong odor that is rich in hydrogen. Other alcohols include cyclohexanol, isobutyl alcohol, or amyl alcohol. The molecular structure of these alcohols is illustrated below and demonstrates the availability of hydrogen for producing oxidizing radicals.

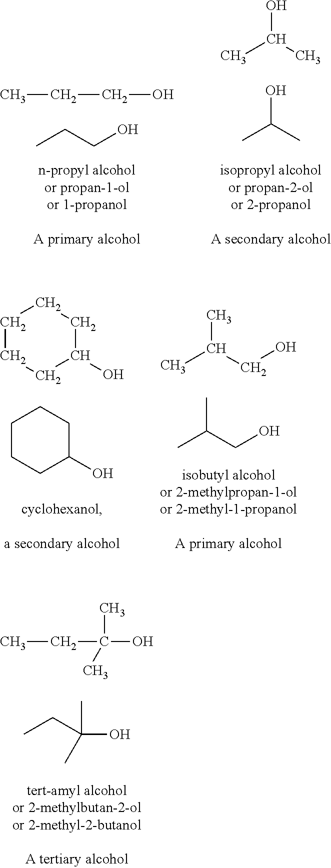

The system 100 described above is a small, lightweight, relatively low cost instrument sterilizer with high throughput and flash sterilization potential. As described herein, used and unsanitary tools or newly manufactured tools are transformed into sterile instruments sealed in a sterile environment for potentially indefinite storage. It requires water only for washing the surgical instruments prior to sterilization, as is required by all instrument sterilization systems, and the water for the presoak is be reusable. The surgical instruments to be sterilized will not need wrapping and are not touched or otherwise exposed to contaminants once the sterilization process is initiated. The system 100 can require as little as 336 watts during use. Furthermore, as inputs to the sterilization process it requires only a supply of tank oxygen and a small supply of isopropyl alcohol (or other secondary molecule), both of which are typically available and needed in a medical setting for other purposes. Isopropyl alcohol can be used in a 68%-99% concentration, in other commercially available concentrations, or a 100% concentration (i.e., pure isopropyl alcohol). In accordance with one advantageous embodiment, isopropyl alcohol can be used in a 70% concentration. Compared to steam autoclaves currently in use, the system provides improvements in capability, throughput, cycle time, electric power and water requirement, the needed supplementary supplies, total weight, size, and cost. Hence, it can be beneficially deployed in mobile or portable settings such as military field hospitals.

The same features that make the device suitable and desirable for military use make it appropriate for public use. Autoclaves are practically ubiquitous in hospitals, nursing homes, operating suites, clinics, animal medical facilities, emergency services, research and development, and testing laboratories. The unit can replace autoclaves, requiring less space and providing more capacity. It will also be useful for surgical instrument manufacturers for factory sterilization of newly manufactured, surgical instruments, and other devices for which sterilization is required.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention. The various functional modules that are shown are for illustrative purposes only, and may be combined, rearranged and/or otherwise modified.

We claim:

1. A system for sterilizing an item comprising:
an ozone source;
a source of a secondary molecule comprising a hydrogen moiety; and
a container for storing the item and configured to be sealed after insertion of at least the item, an amount of the secondary molecule, and a volume of ozone.

2. The system of claim 1, further comprising a vacuum pump for at least partially evacuating the container prior to insertion of the ozone.

3. The system of claim 1, wherein the ozone source comprises an ozone generator and a storage unit for storing a gas output from the ozone source.

4. The system of claim 1, wherein the ozone generator is configured to output a mixture of ozone and oxygen.

5. The system of claim 4, wherein the mixture of ozone and oxygen comprises approximately 7.5% partial pressure of ozone.

6. The system of claim 1, wherein the secondary molecule comprises a liquid, the system further comprising an absorbent article for conveying the secondary molecule into the container.

7. The system of claim 6, wherein the container comprises the absorbent article.

8. The system of claim 1, wherein the secondary molecule comprises an alcohol.

9. The system of claim 8, wherein the alcohol comprises isopropyl alcohol.

10. The system of claim 1, further comprising a pre-soak unit for soaking the item in a heated water-based solution prior to insertion in the container.

11. The system of claim 10, wherein the pre-soak unit is configured to pressurize the heated water-based solution.

12. The system of claim 10, wherein the water-based solution includes nutrients for encouraging germination of spores on the item.

13. The system of claim 1, further comprising a catalyst attached to a release valve such that when the release valve is opened, a gas mixture contained in the container passes through the carbon catalyst.

14. A method of sterilizing an item comprising:
inserting a secondary molecule comprising a hydrogen moiety into a container storing the item to be sterilized;
inserting a gaseous mixture comprising ozone into the container; and
sealing the container.

15. The method of claim 14, further comprising applying at least a partial vacuum to the container prior to filling the container with the gaseous mixture.

16. The method of claim 14, wherein the gaseous mixture further comprises oxygen.

17. The method of claim 16, wherein the gaseous mixture comprises approximately 7.5% partial pressure of ozone.

18. The method of claim 14, wherein the secondary molecule comprises a liquid and the secondary molecule is inserted into the container via an absorbent article.

19. The method of claim 18, wherein the container comprises the absorbent article.

20. The method of claim 14, wherein the secondary molecule comprises an alcohol.

21. The method of claim 20, wherein the alcohol comprises isopropyl alcohol.

22. The method of claim 14, further comprising soaking the item in a heated water-based solution prior to insertion in the container.

23. The method of claim 22, further comprising pressurizing the heated water-based solution.

24. The method of claim 23, wherein the water-based solution includes nutrients for encouraging germination of spores on the item.

25. The method of claim 14, wherein the secondary molecule is inserted into the container as a vapor.

26. A method of sterilizing an item comprising:
exposing the item to a gaseous mixture comprising ozone and a secondary molecule comprising a hydrogen moiety.

27. The method of claim 26, further comprising storing the item in a container.

28. The method of claim 26, further comprising applying at least a partial vacuum to a container storing the item prior to exposing the item to the secondary molecule and the gaseous mixture.

29. The method of claim 26, wherein the gaseous mixture comprises approximately 7.5% partial pressure of ozone.

30. The method of claim 26, wherein the secondary molecule comprises an alcohol.

31. The method of claim 30, wherein the alcohol comprises isopropyl alcohol.
